(19) United States
(12) Patent Application Publication
(10) Pub. No.: US 2006/0276745 A1
(43) Pub. Date: Dec. 7, 2006

(54) Manual breast pump

(76) Inventor: Kun-Chung Tsai, Yung Kang City (TW)

Correspondence Address: LEONG C LEI, PMB # 1008, 1867 YGNACIO VALLEY ROAD, WALNUT CREEK, CA, 94598, US

(21) Appl. No.: 11/142,297

(22) Filed: Jun. 2, 2005

(51) Int. Cl. A61M 1/06

(52) U.S. Cl. 604/74

(57) ABSTRACT

A manual breast pump can be operated by one hand is provided. Urging a funnel against a human breast and squeezing a flexible handle to press an arm will straighten the arm to lift a sliding member along two opposite grooves of a support and lift a piston respect to a cylinder so as to create a vacuum in a portion of the cylinder below a piston ring and the vacuum is communicated to the funnel for drawing milk from the breast into a blocked valve on a top opening of a bottle through the funnel for temporarily storing. Immediately releasing the handle will pivot both links of the arm toward each other, move both the sliding member and the piston downwardly to exert a force on the valve such that the valve is open to drop the milk into the bottle.

### BACKGROUND OF THE INVENTION

(a) Technical Field of the Invention

The present invention relates to breast pumps and more particularly to a manually operated breast pump which can be operated by one hand.

(b) Description of the Prior Art

Nursing mothers often use mechanized breast pumps to express milk from their breasts for bottle-feeding their infant children. Pumped milk must be refrigerated because often it will not be immediately consumed. A conventional manual breast pump is shown in FIG. 1. It comprises a forward funnel 70 for receiving a human breast, a rear flexible bulb 71, a tube 701 interconnected the funnel 70 and the bulb 71, and a reservoir 702 on a bottom of the tube 701. In use, releasing the pressed bulb 71 will create a vacuum to draw milk into the reservoir 702 via the tube 701. Its drawback is that the reservoir 702 is too small to contain much pumped milk. As such, an additional large bottle is required to contain milk poured from the full reservoir 702. Such frequent pouring may contaminate the milk.

Another conventional breast pump is shown in FIG. 2. It comprises a forward funnel 80, a rear flexible bulb 82, a funnel-supporting shield neck 801 provided in a rear of the funnel 80, a hose 821 interconnected the shield neck 801 and the bulb 82 and in fluid communication therewith, and a bottle 81 coupled to both the shield neck 801 and the funnel 80. In use, one hand of a mother holds the bottle 81 to receive and support the mother's breast in the funnel 80 and the other hand thereof is used to reciprocate the bulb 82. However, the other hand of the mother is also required to encourage lactation. Such frequent change of the other hand may cause the milk pumping operation to be out of order. Thus, the need for improvement still exists.

### SUMMARY OF THE INVENTION

It is an object of the present invention to provide a manually operated breast pump which can be operated by one hand such that milk pumping operation can be more orderly. Also, components of the breast pump can be easily disassembled for cleaning and sanitizing.

To achieve the above and other objects, the present invention provides a manual breast pump comprising a bottle including an annular seal lip formed on a top opening and a valve formed in a center of the seal lip; a funnel including a breast shield for receiving a human breast and a cylindrical shield neck having an annular, hollow cap threaded and secured to the top opening of the bottle; a support including an upright shroud, a pivot pin on a lower, outer portion of the shroud, two opposite latches extended downwardly from the shroud to fasten at an outer periphery of the cap, two opposite posts extended from the shroud, two lengthwise grooves each formed on an inner surface of the post, and two cavities each formed on a top of the post; a cylinder including an annular detent projected downwardly from a bottom to fasten at an inner periphery of the cap so as to join closely with the shroud; a piston received in a sliding manner with the cylinder which includes a bottom piston ring and a top attachment member; a member which slides along the grooves which also includes a top transverse bar and a bottom recess which snaps secured to the attachment member; a hinge member including a top sleeve and two bottom hooks fastened in the cavities; an arm including an upper link having a C-shaped top pivotable slot placed on the bar, a lower link having a C-shaped bottom pivotable slot placed on the pin, and an intermediate spring pivoted to both the upper link and the lower link; and a flexible handle for enclosing the arm, including a pivotable pin which is inserted into the sleeve so as to be hinged coupled thereto and an resilient member extended downwardly from an inner surface to engage with the arm; whereby: urging the breast shield against a human breast and squeezing the handle to press the resilient member on the arm will straighten the arm to lift the sliding member along the grooves and lift the piston respect to the cylinder so as to create a vacuum in a portion of the cylinder below the piston ring and the vacuum is communicated to the funnel for drawing milk from the breast into the blocked valve through the funnel and the seal lip for temporarily storing; and releasing the handle will pivot the upper link and the lower link toward each other, move both the sliding member and the piston downwardly to exert a force on the valve such that the valve is open to drop the milk into the bottle.

The foregoing object and summary provide only a brief introduction to the present invention. To fully appreciate these and other objects of the present invention as well as the invention itself, all of which will become apparent to those skilled in the art, the following detailed description of the invention and the claims should be read in conjunction with the accompanying drawings. Throughout the specification and drawings identical reference numerals refer to identical or similar parts.

Many other advantages and features of the present invention will become manifest to those versed in the art upon making reference to the detailed description and the accompanying sheets of drawings in which a preferred structural embodiment incorporating the principles of the present invention is shown by way of illustrative example.

### BRIEF DESCRIPTION OF THE DRAWINGS

FIG. 1 is a side view of a conventional manual breast pump;

FIG. 2 is a side view of another conventional manual breast pump;

FIGS. 3 and 4 are exploded views of a preferred embodiment of manual breast pump according to the invention;

FIG. 5 is a partially assembled view of the breast pump shown in FIG. 4

FIG. 6 is a perspective view of the assembled components shown in FIG. 5 to be fully assembled with two mated portions of the handle; and

FIGS. 7 and 8 are schematic side views in part section for illustrating the milk expressing operation of the invention.

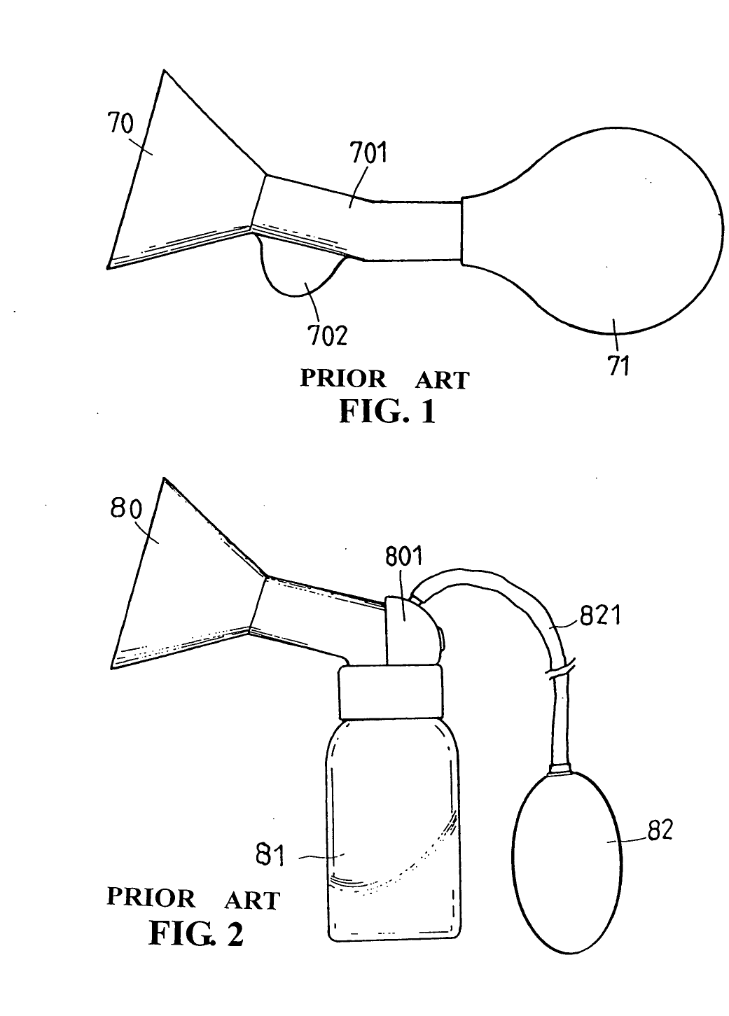

### DETAILED DESCRIPTION OF THE PREFERRED EMBODIMENTS

The following descriptions are of exemplary embodiments only, and are not intended to limit the scope, applicability or configuration of the invention in any way. Rather, the following description provides a convenient illustration for implementing exemplary embodiments of the invention. Various changes to the described embodiments may be made in the function and arrangement of the elements described without departing from the scope of the invention as set forth in the appended claims.

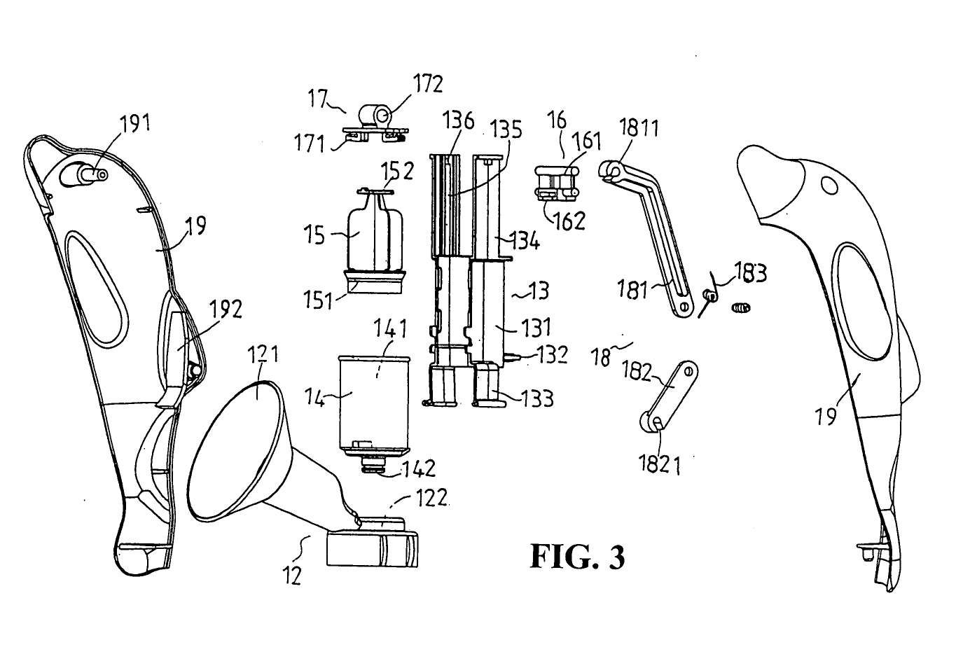

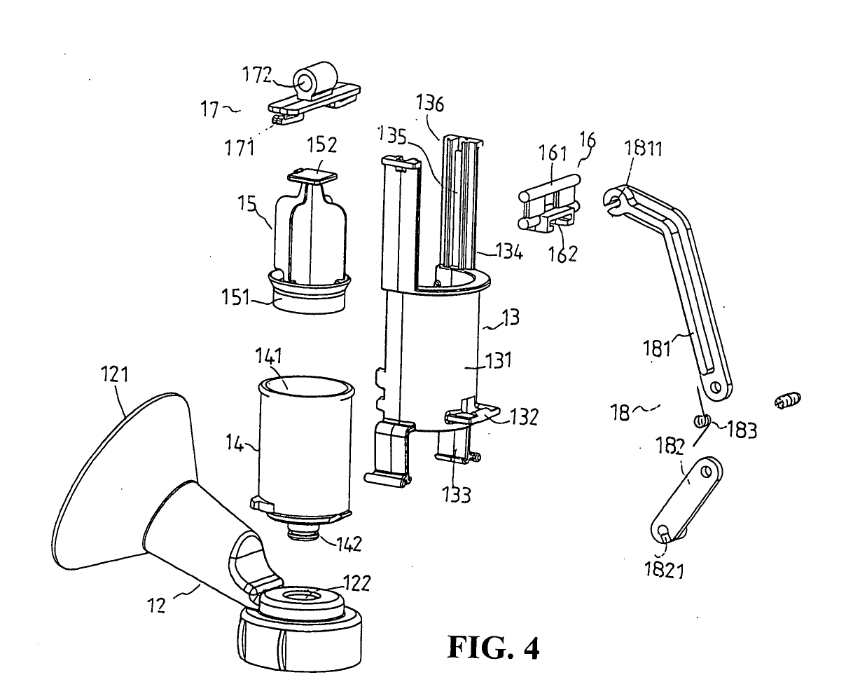

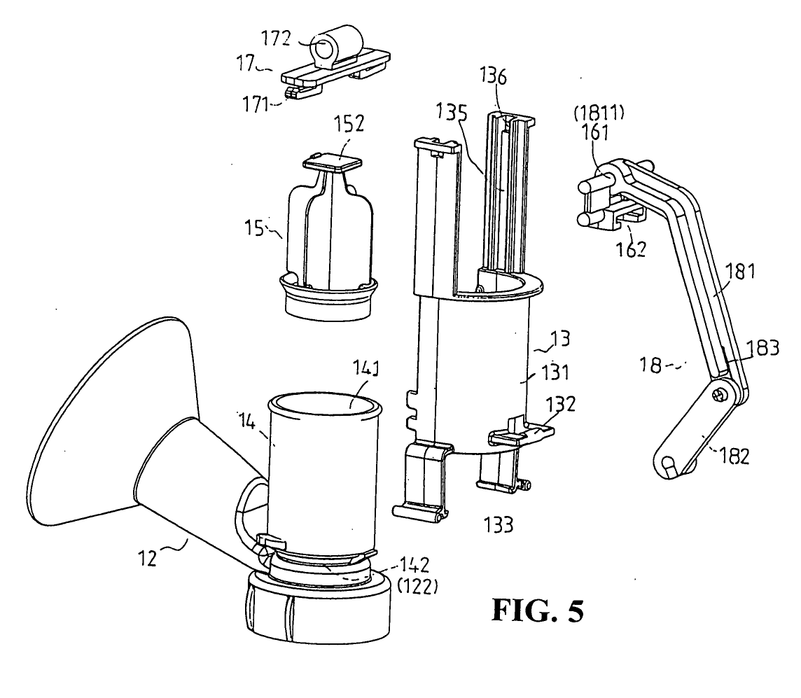

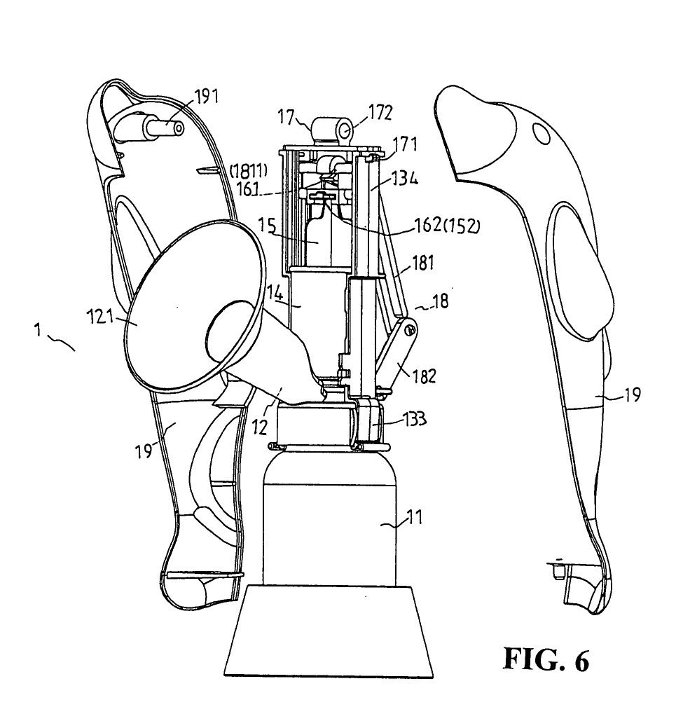

Referring to FIGS. 3 to 8, there is shown a manual breast pump 1 constructed in accordance with a preferred embodiment of the invention comprising a bottle 11, a funnel 12, a support 13, a cylinder 14, a piston 15, a sliding member 16, a hinge member 17, an arm 18, and a handle 19. Each component will be described in detailed below.

The bottle 11 comprises an annular seal lip 110 formed on a top opening and a valve 111 formed in the center of the seal lip 110. The funnel 12 comprises a breast shield 121 for receiving a human breast and a cylindrical shield neck having an annular, hollow cap 122 threaded to the top opening of the bottle 11. The support 13 comprises an upright shroud 131 having a half-circular section, a pivot pin 132 on a lower, outer portion of the shroud 131, two opposite legs 133 having a latched end extended downwardly from the shroud 131 to fasten at an outer periphery of the cap 122, two opposite posts 134 extended upwardly from the shroud 131, a lengthwise groove 135 formed on an inner surface of each post 134, and a cavity 136 formed on a top of each post 135.

The cylinder 14 comprises a bore 141 and an annular detent 142 projected downwardly from a bottom to fasten at an opening of the cap 122. At this position, the shroud 131 is put on the cylinder 14. The piston 15 is slid into the bore 141 of the cylinder 14 and comprises a bottom piston ring 151 and a top attachment member 152. The sliding member 16 is slid along the grooves 135 and comprises a top transverse bar 161 and a bottom recess 162 which is snapped and secured to the attachment member 152. The elongated hinge member 17 comprises a top sleeve 172 and two bottom hooks 171 fastened in the cavities 136. The aim 18 comprises an upper link 181 having a C-shaped top pivotable slot 1811 put on the bar 161, a lower link 182 having a C-shaped bottom pivotable slot 1821 put on the pin 132, and an intermediate pivotable coil spring 183 attached to both a bottom hole of the upper link 181 and a top hole of the lower link 182. The flexible handle 19 for enclosing the arm 18 and comprises two joined portions each including a pivotable pin 191 inserted into the sleeve 172 (i.e., hinged together) and a leaf-spring finger 85 extended downwardly from an inner surface of the handle 19 to engage with the arm 18.

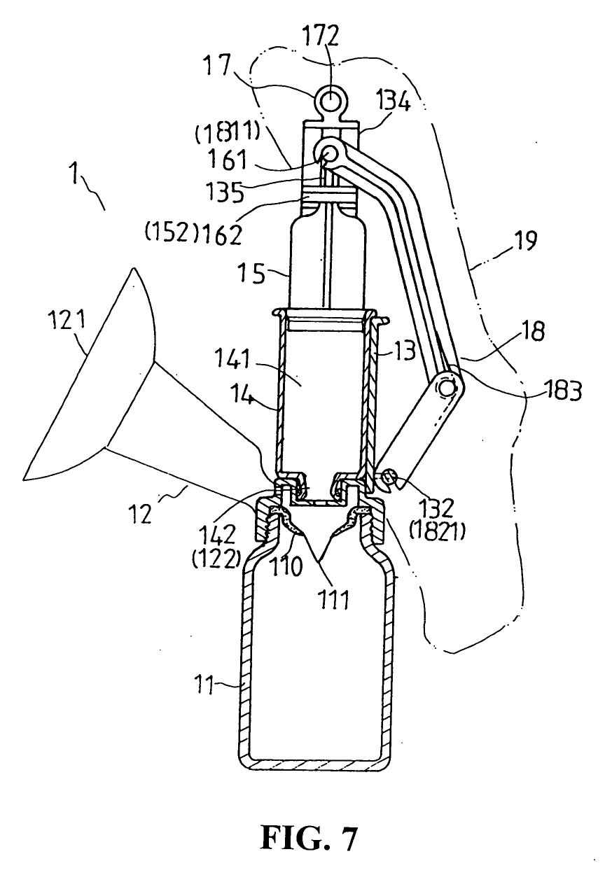

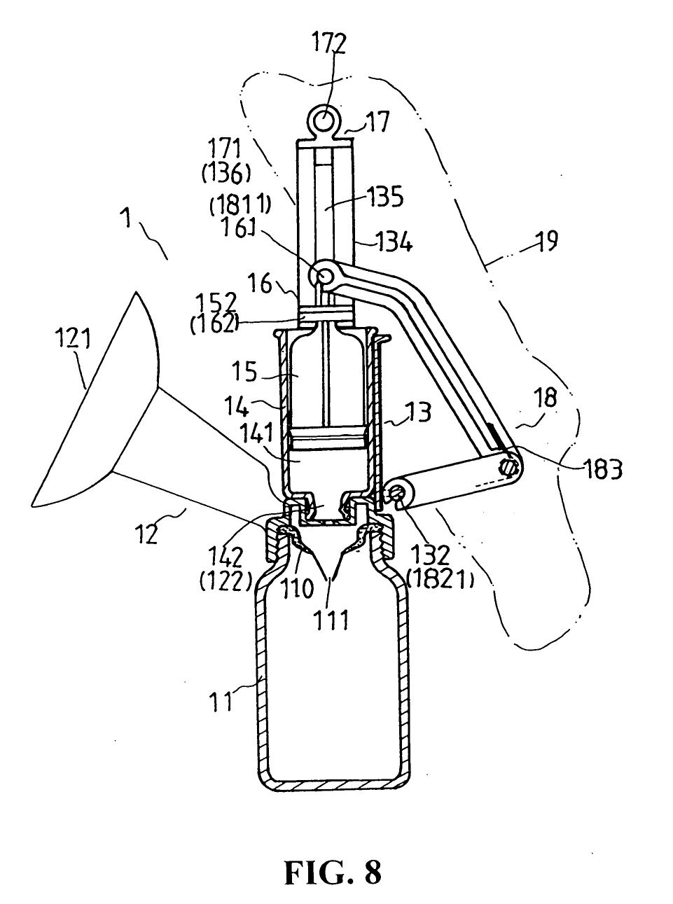

Referring to FIGS. 7 and 8 specifically, a milk expressing operation of the invention will be described in detail below. A mother uses one hand to urge the breast shield 121 against her breast and the other hand to squeeze the handle 19 to press the finger 85 on the arm 18. The upper link 181 and the lower link 182 then straighten to lift the sliding member 16 along the grooves 134 due to the linking mechanism of the upper link 181 and the sliding member 16. And in turn, the piston 15 lifts respect to the bore 141 of the cylinder 14. A vacuum is thus created in a chamber confined by the piston ring 151 and a bottom of the cylinder 14. The vacuum is communicated through the cap 122 to the funnel 12 such that milk is drawn into the funnel 12. The milk flows from the funnel 12 through the cap 122 and, due to the configuration, drops through the seal lip 110 into the valve 111 for temporarily storing because the valve 111 is blocked (see FIG. 7). A release of the handle 19 will pivot the upper link 181 and the lower link 182 toward each other by pivoting about the spring 183. And in turn, the sliding member 16 moves downwardly along the grooves 134 due to the linking mechanism of the upper link 181 and the sliding member 16. The piston 15 then lowers respect to the bore 141 of the cylinder 14. A pressure is thus exerted on the valve 111 by the chamber confined by the piston ring 151 and the bottom of the cylinder 14. The valve 111 is thus open to drop the milk into the bottle 11 (see FIG. 8). By reciprocating as above, milk can be stored in the bottle 11 by one hand operation in a simple, convenient way. Moreover, components of the breast pump 1 can be easily disassembled for cleaning.

It will be understood that each of the elements described above, or two or more together may also find a useful application in other types of methods differing from the type described above.

While certain novel features of this invention have been shown and described and are pointed out in the annexed claim, it is not intended to be limited to the details above, since it will be understood that various omissions, modifications, substitutions and changes in the forms and details of the device illustrated and in its operation can be made by those skilled in the art without departing in any way from the spirit of the present invention.

I claim:

1. A manual breast pump comprising:
a bottle including an annular seal lip formed on a top opening and a valve formed in a center of the seal lip;
a funnel including a breast shield for receiving a human breast and a cylindrical shield neck having an annular, hollow cap release-secured to the top opening of the bottle;
a support including an upright shroud, a pivot pin on a lower, outer portion of the shroud, two opposite latches extended downwardly from the shroud to fasten at an outer periphery of the cap, two opposite posts extended from the shroud, two lengthwise grooves each formed on an inner surface of the post, and two cavities each formed on a top of the post;
a cylinder including an annular detent projected downwardly from a bottom to fasten at an inner periphery of the cap so as to joined with the shroud;
a slidable piston received in the cylinder and including a bottom piston ring and a top attachment member;
a member slidable along the grooves and including a top transverse bar and a bottom recess snapped and secured to the attachment member;
a hinge member including a top sleeve and two bottom hooks fastened in the cavities;
an arm including an upper link having a C-shaped pivotable top slot put on the bar, a lower link having a C-shaped bottom pivotable slot put on the pin, and an intermediate pivotable spring joined to both the upper link and the lower link; and
a flexible handle for enclosing the arm and including a pivotable pin inserted into the sleeve so as to be hinged and joined thereto and an resilient member extended downwardly from an inner surface to engage with the arm; whereby:
urging the breast shield against a human breast and squeezing the handle to press the resilient member on the arm will straighten the arm to lift the sliding member along the grooves and lift the piston respect to the cylinder so as to create a vacuum in a portion of the cylinder below the piston ring and the vacuum is communicated to the funnel for drawing milk from the breast into the blocked valve through the funnel and the seal lip for temporarily storing; and
releasing the handle will pivot the upper link and the lower link toward each other, move both the sliding member and the piston downwardly to exert a force on the valve such that the valve is open to drop the milk into the bottle.

2. The manual breast pump of claim 1, wherein the resilient member is a leaf-spring finger.
